(19) United States
(12) Patent Application Publication
(10) Pub. No.: US 2020/0171225 A1
(43) Pub. Date: Jun. 4, 2020

(54) THROMBUS CLEARING MANIFOLD FOR VENTRICULAR ASSIST DEVICES

(71) Applicant: HeartWare, Inc., Miami Lakes, FL (US)

(72) Inventors: David SCHAFIR, Miami Shores, FL (US); Mustafa Ertan TASKIN, Cooper City, FL (US)

(21) Appl. No.: 16/653,231

(22) Filed: Oct. 15, 2019

Related U.S. Application Data
(60) Provisional application No. 62/772,664, filed on Nov. 29, 2018.

(51) Int. Cl. A61M 1/10

(52) U.S. Cl. CPC A61M 1/1086; A61M 1/101; A61M 1/1008

(57) ABSTRACT

A thrombus clearing implantable blood pump including a housing having an inlet cannula and an outlet opposite the inlet cannula, the inlet cannula and the outlet defining a flow path therebetween, and the inlet cannula having a sidewall defining a circumference and a plurality of apertures extending through the sidewall. A rotor and a stator are disposed within the housing for rotating the rotor when a current is applied to the stator.

### CROSS-REFERENCE TO RELATED APPLICATION

This application claims the benefit of U.S. application Ser. No. 62/772,664, filed Nov. 29, 2018

### FIELD

The present technology is generally related to implantable blood pumps.

### BACKGROUND

Mechanical Circulatory Support Devices (“MCSDs”), such as ventricular assist devices, are commonly used to assist the pumping action of a failing heart. Typically, an MCSD is surgically implanted in a patient's body and includes a housing with an inlet, an outlet, and a rotor mounted therein. The inlet is connected to a chamber of the patient's heart, typically the left ventricle, whereas the outlet is connected to an artery, such as the aorta. Rotation of the rotor drives blood from the inlet towards the outlet and thus assists blood flow from the chamber of the heart into the artery.

Blood pumps used in MCSDs are desirably provided with contactless bearings so that the rotor floats within the housing in operation. With contactless bearings, there is no solid-to-solid contact between the rotor and the housing, and thus no mechanical wear during operation. One form of contactless bearing is a hydrodynamic bearing. In a hydrodynamic bearing, the liquid being pumped passes between a surface of the rotor and the surfaces of a hydrodynamic bearing which creates a clearance that is many times larger than the dimensions of blood cells. The surfaces are configured so that as the rotor turns, the fluid disposed between these surfaces exerts pressure on the surface of the rotor that holds the rotor away from the housing. However, in some cases the blood passing through the blood pump may contain particles that lead to thrombus, a solid or semi-solid deposit generated within a patient's body. The thrombus can lodge on the surface of the hydrodynamic bearing and impede its operation, which is hazardous or lethal for the patient. Unfortunately, known blood pumps fail to include a system for dislodging and/or removing the thrombus, or other harmful particles, from the blood pump.

### SUMMARY

The techniques of this disclosure generally relate to an implantable blood pump configured to clear a thrombus from the blood pump.

In one aspect, the present disclosure provides a thrombus clearing implantable blood pump including a housing having an inlet cannula and an outlet opposite the inlet cannula, the inlet cannula and the outlet defining a flow path therebetween, and the inlet cannula having a sidewall defining a circumference and a plurality of apertures extending through the sidewall; a rotor disposed within the housing; and a stator disposed within the housing for rotating the rotor when a current is applied to the stator.

In another aspect, the disclosure provides apertures defining a thrombus exit region from within the flow path to outside of the housing.

In another aspect, the disclosure provides the rotor and the stator defining a centrifugal force for expelling a thrombus through at least one of the plurality of apertures when the thrombus is within the blood pump.

In another aspect, the disclosure provides the inlet cannula including a proximal portion and a distal portion opposite the proximal portion, the distal portion being proximate the outlet, and the proximal portion defining the apertures.

In another aspect, the disclosure provides the blood pump including an inner tube disposed within the inlet cannula, and the proximal portion of the inlet cannula extending beyond the inner tube away from the outlet.

In another aspect, the disclosure provides the apertures extending around at least a portion of the circumference of the sidewall.

In another aspect, the disclosure provides the apertures including a first set of apertures and a second set of apertures, the second set of apertures being disposed parallel with respect to the first set of apertures.

In another aspect, the disclosure provides the apertures including a circular portion and an elongated portion extending from the circular portion.

In one aspect, the present disclosure provides a thrombus clearing implantable blood pump including a housing having an inlet cannula and an outlet, the inlet cannula and the outlet defining a flow path therebetween, and the inlet cannula including a sidewall defining a circumference of the flow path and an aperture extending through the sidewall transverse to the flow path; a rotor disposed within the inlet cannula of the housing; a stator disposed within the housing for rotating the rotor when a current is applied to the stator.

In another aspect, the disclosure provides the rotor and the stator defining a centrifugal force for expelling a thrombus through the aperture when the thrombus is within the blood pump.

In another aspect, the disclosure provides the aperture including a circular portion and an elongated portion extending from the circular portion and around a portion of the sidewall.

In another aspect, the disclosure provides the inlet cannula including a proximal portion and a distal portion opposite the proximal portion, the distal portion being proximate the outlet, and the proximal portion defining the aperture.

In another aspect, the disclosure provides an inner tube disposed within the inlet cannula and being sized to receive the rotor therein, and the proximal portion of the inlet cannula extending beyond the inner tube.

In another aspect, the disclosure provides one or more apertures extending around the circumference of the sidewall and including a first set of apertures and a second set of apertures arranged in a parallel orientation with respect to each other.

In one aspect, the present disclosure provides a thrombus clearing implantable blood pump including a housing including an inlet cannula having a cylindrical portion and a curved portion extending from the cylindrical portion, the curved portion defining a thrombus outlet facing away from the curved portion; a chamber coupled to the cylindrical portion of the inlet cannula, the chamber defining a fluid outlet, and the inlet cannula and the fluid outlet defining a fluid flow path therebetween; and a rotor disposed within the chamber.

In another aspect, the disclosure provides the rotor and the curved portion of the inlet cannula defining a centrifugal force for ejecting a thrombus when disposed within the housing through the thrombus outlet.

In another aspect, the disclosure provides the thrombus outlet being transverse to the cylindrical portion of the inlet cannula.

In another aspect, the disclosure provides the thrombus outlet including an extension member extending from the inlet cannula and defining a thrombus aperture.

In another aspect, the disclosure provides the curved portion of the inlet cannula defining an inlet facing away from the thrombus outlet.

In one aspect, the present disclosure provides a thrombus clearing implantable blood pump including a housing having an inlet cannula and an outlet opposite the inlet cannula, the inlet cannula and the outlet defining a flow path therebetween, and the inlet cannula including a sidewall defining a circumference, a proximal portion, and a distal portion opposite the proximal portion, the distal portion being proximate the outlet, and the proximal portion defining a plurality of apertures extending through the sidewall and around the circumference; an inner tube disposed within the inlet cannula, and the proximal portion of the inlet cannula extending beyond the inner tube away from the outlet; and a rotor and a stator disposed within the housing, the stator being configured to rotate the rotor when a current is applied to the stator, the rotor and the stator defining a centrifugal force for expelling a thrombus through at least one of the plurality of apertures when the thrombus is within the blood pump.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of a thrombus clearing implantable blood pump that illustrates an inlet cannula having a sidewall defining a circumference and one or more apertures extending through the sidewall;

FIG. 2 is a cross-sectional perspective view of the blood pump taken along section A-A of FIG. 1 that illustrates a rotor within the inlet cannula;

FIG. 3 is an enlarged partial perspective view of the inlet cannula of the blood pump FIG. 1 that illustrates the apertures including an extension member extending therefrom;

FIG. 4 is an enlarged partial perspective view of the inlet cannula of the blood pump FIG. 1 that illustrates the extension member extending from the aperture; and

FIG. 5 is a perspective view of another embodiment of a thrombus clearing implantable blood pump.

### DETAILED DESCRIPTION

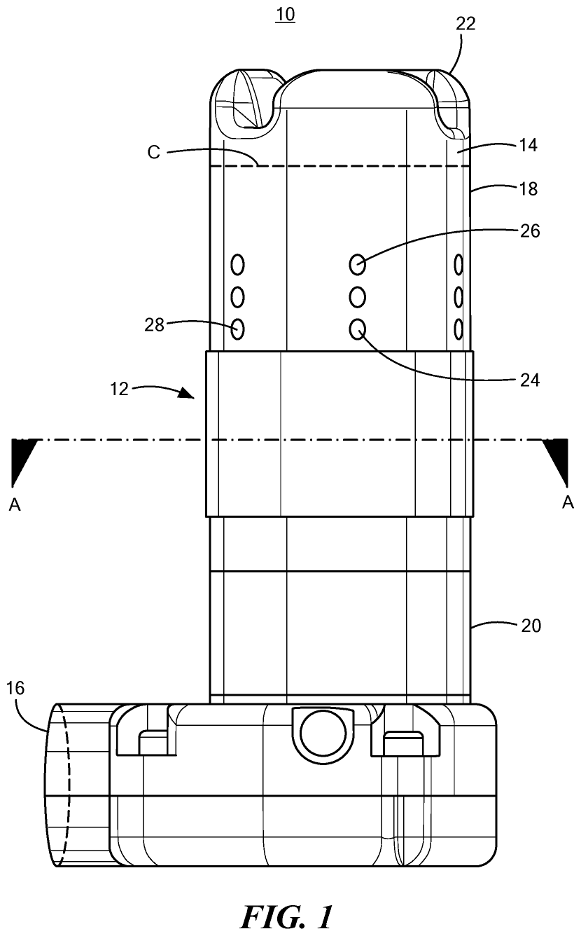

Referring now to FIGS. 1-4 an exemplary thrombus clearing implantable blood pump is provided and designated generally as “10”. The blood pump 10 is configured to be implanted in a patient, such as a human or animal patient, to assist in circulating blood from a heart of the patient to the rest of the body. The blood pump 10 may operate similar to the design features of the blood pump manufactured and sold under the designation MVAD®. During operation, the blood pump 10 is configured to eject a thrombus from the blood pump when present therein, as discussed below.

FIG. 1 is a perspective view of the blood pump 10 including a housing 12 having an inlet cannula 14 and an outlet 16 opposite the inlet cannula 14. The inlet cannula 14 and the outlet 16 define a flow path therebetween for a fluid, such as blood, to flow through the blood pump 10. The inlet cannula 14 includes a proximal portion 18 and a distal portion 20 opposite the proximal portion 18, the distal portion 20 being proximate the outlet 16. The inlet cannula 14 also includes a sidewall 22 defining a circumference “C” and one or more apertures 24 extending through the sidewall 22 at the proximation portion 18. The circumference of the sidewall 22 at least partially defines the circumference of the flow path through the inlet cannula 14. The number of apertures 24 may vary in accordance with various design configurations. The apertures 24 define a thrombus exit region from within the flow path to outside of the housing 12 when a thrombus is present within the blood pump 10. The apertures 24 may also allow other hazardous particles to be expelled from the blood pump 10 in the manner described with respect to the thrombus.

The apertures 24 extend around at least a portion of the circumference of the sidewall 22. In one configuration, the apertures 24 extend around the entire circumference of the sidewall 22 and may be evenly or non-evenly spaced from each other. In other configurations, the apertures 24 extend less than the entire circumference, such as between 25% to 75% of the circumference, and various configurations may be provided. FIG. 1 depicts the apertures 24 including a first set of apertures 26 and a second set of apertures 28, the second set of apertures 28 being disposed parallel with respect to the first set of apertures 26. The apertures 24 are depicted as being generally circular, but may be provided in various shapes, such as oval, square, etc.

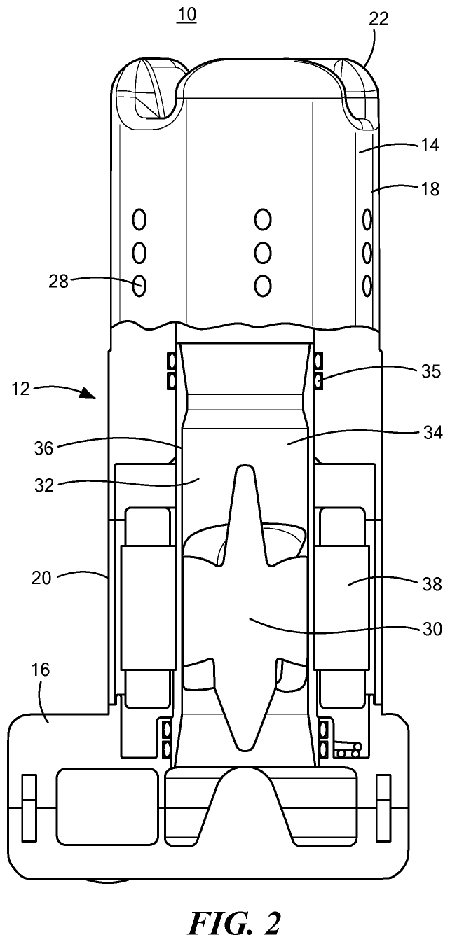

FIG. 2 is a cross-sectional perspective view of the blood pump taken along section A-A of FIG. 1 that depicts a rotor 30 disposed within the housing 12 and proximate the inlet cannula 14 to impel the blood. In particular, the inlet cannula 14 may include an inner tube 32 formed from a non-magnetic material, such as a ceramic, which defines a bore 34 and the rotor 30 may be disposed within the bore 34. The inner tube 32 may be coupled to the inlet cannula 14 using one or more O-ring grooves 35 which form a seal therebetween. The proximal portion 18 of the inlet cannula 14 extends beyond the inner tube 32 away from the outlet 16 so as not to obstruct the apertures 24.

The inner tube 32 includes a cylindrical outer surface 36 surrounded by a stator 38 disposed within the housing 12 for rotating the rotor 30 when an electrical current is applied to the stator 38 from a drive circuit (not shown). The rotor 30 and the stator 38 define a centrifugal force for expelling the thrombus through at least one of the apertures 24 when the thrombus is within the blood pump 10. For example, assuming there is circulating thrombus in a patient's bloodstream, the blood pump 10 is configured to utilize the energy of the flow boundary layer of the blood in addition to the centrifugal forces in the flow blood flow to eject the thrombus through the apertures in the inlet cannula 14.

FIG. 2 depicts the inlet cannula 14 defining an extension member 40 extending from the aperture 24 to guide the thrombus through the aperture 24 and/or the extension member 40. In other words, the apertures 24 may include a circular portion and an elongated portion, such as the extension member 40, extending from the circular portion.

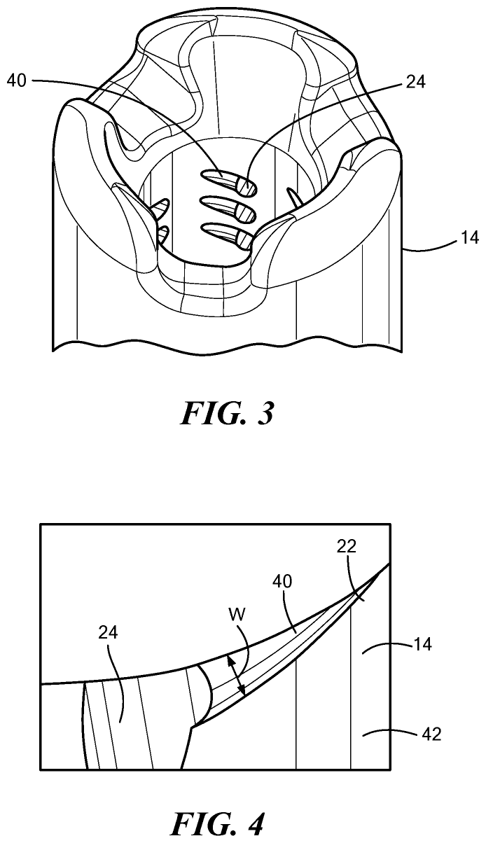

FIG. 3 is an enlarged partial perspective view of the inlet cannula 14 defining the aperture 24 and the extension member 40 extending therefrom. The extension member 40 may be provided in various shapes, such as oval, circular, and the like. The length of the extension member 40 may vary in accordance with the dimensions of the inlet cannula 14.

FIG. 4 is an enlarged partial perspective view of the aperture 24 and the extension member 40 sloping in a direction toward the aperture 24 similar to the form of a ramp. For example, the extension member 40 defines a width “W” which slopes in a direction toward an interior portion 42 of the sidewall 22 of the inlet cannula 14.

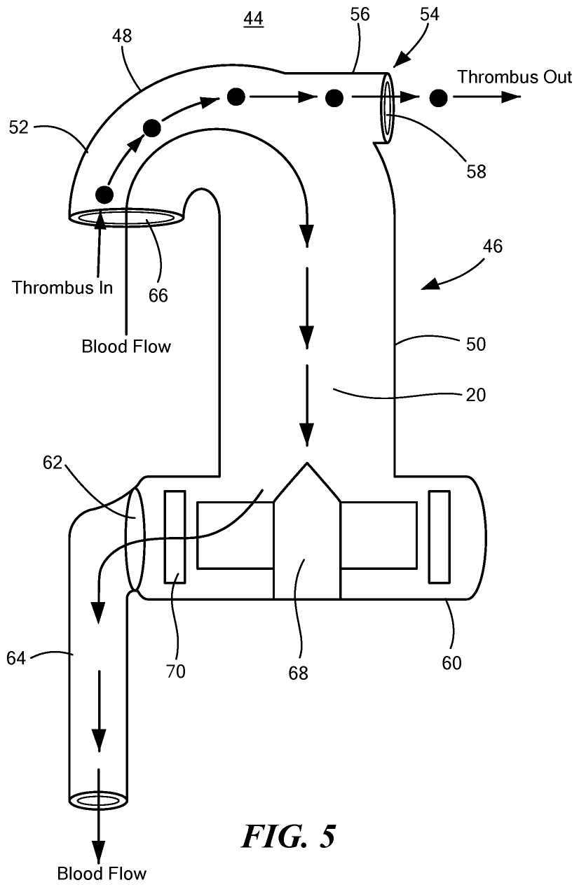

FIG. 5 is a perspective view of another embodiment of a thrombus clearing implantable blood pump generally designated as “44”. The blood pump 44 includes a housing 46 having an inlet cannula 48 including a cylindrical portion 50 and a curved portion 52 extending from the cylindrical portion 50. The curved portion 52 defines a thrombus outlet 54 facing away from the curved portion 52 and transverse to the cylindrical portion 50 of the inlet cannula 48. In one configuration, the thrombus outlet 54 includes an extension member 56 extending from the inlet cannula 48 and defining a thrombus aperture 58. The thrombus outlet 54 defines a path to expel a thrombus, or another harmful or foreign particle, from within the blood pump 44 to outside of the blood pump 44 through the thrombus aperture 58.

A chamber 60 is coupled to the cylindrical portion 50 of the inlet cannula 48. The chamber 60 defines a fluid outlet 62 for fluid to exit the blood pump 44 and a graft 64 may be coupled to the chamber 60 such that fluid exits the fluid outlet 62 and enters the graft 64. The curved portion 52 of the inlet cannula 48 also defines an inlet 66 facing away from the thrombus outlet 54 and fluid enters the blood pump 44 through the inlet 66. As such, the inlet cannula 48 and the fluid outlet 62 define a fluid flow path therebetween through the blood pump 44.

A rotor 68 is disposed within the chamber 60 in communication with a stator 70 disposed within the housing 46 for rotating the rotor 68 when an electrical current is applied to the stator 70 from a drive circuit (not shown). The rotor 68 and the curved portion 52 of the inlet cannula 48 define a centrifugal force for ejecting the thrombus through the thrombus outlet 54 and outside of the blood pump 44 when the thrombus is present within the housing 46. The thrombus outlet 54 may also allow the harmful or foreign particles to be expelled from the blood pump 44 in the manner described with respect to the thrombus. The curvature of the curved portion 52 of the inflow cannula 48 assists in generating the centrifugal forces that separate the thrombus having a relatively heavier weight than other portions of the blood from the blood flow for discharge through the thrombus outlet 54.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A thrombus clearing implantable blood pump comprising:
a housing including:
an inlet cannula and an outlet opposite the inlet cannula, the inlet cannula and the outlet defining a flow path therebetween, and the inlet cannula having a sidewall defining a circumference and a plurality of apertures extending through the sidewall;
a rotor disposed within the housing; and
a stator disposed within the housing for rotating the rotor when a current is applied to the stator.

2. The blood pump of claim 1, wherein the plurality of apertures define a thrombus exit region from within the flow path to outside of the housing.

3. The blood pump of claim 1, wherein the rotor and the stator define a centrifugal force for expelling a thrombus through at least one of the plurality of apertures when the thrombus is within the blood pump.

4. The blood pump of claim 1, wherein the inlet cannula includes a proximal portion and a distal portion opposite the proximal portion, the distal portion being proximate the outlet, and the proximal portion defining the plurality of apertures.

5. The blood pump of claim 4, further comprising an inner tube disposed within the inlet cannula, and the proximal portion of the inlet cannula extending beyond the inner tube away from the outlet.

6. The blood pump of claim 1, wherein the plurality of apertures extend around at least a portion of the circumference of the sidewall.

7. The blood pump of claim 1, wherein the plurality of apertures include a first set of apertures and a second set of apertures, the second set of apertures being disposed parallel with respect to the first set of apertures.

8. The blood pump of claim 1, wherein the plurality of apertures includes a circular portion and an elongated portion extending from the circular portion.

9. A thrombus clearing implantable blood pump comprising:
a housing including:
an inlet cannula and an outlet, the inlet cannula and the outlet defining a flow path therebetween, and the inlet cannula including a sidewall defining a circumference of the flow path and an aperture extending through the sidewall transverse to the flow path;
a rotor disposed within the inlet cannula of the housing; and
a stator disposed within the housing for rotating the rotor when a current is applied to the stator.

10. The blood pump of claim 9, wherein the rotor and the stator define a centrifugal force for expelling a thrombus through the aperture when the thrombus is within the blood pump.

11. The blood pump of claim 9, wherein the aperture includes a circular portion and an elongated portion extending from the circular portion and around a portion of the sidewall.

12. The blood pump of claim 9, wherein the inlet cannula includes a proximal portion and a distal portion opposite the proximal portion, the distal portion being proximate the outlet, and the proximal portion defining the aperture.

13. The blood pump of claim 12, further comprising an inner tube disposed within the inlet cannula and being sized to receive the rotor therein, and the proximal portion of the inlet cannula extending beyond the inner tube.

14. The blood pump of claim 9, further comprising a plurality of apertures extending around the circumference of the sidewall and including a first set of apertures and a second set of apertures arranged in a parallel orientation with respect to each other.

15. A thrombus clearing implantable blood pump comprising:
a housing including:
an inlet cannula having a cylindrical portion and a curved portion extending from the cylindrical portion, the curved portion defining a thrombus outlet facing away from the curved portion;
a chamber coupled to the cylindrical portion of the inlet cannula, the chamber defining a fluid outlet, and the inlet cannula and the fluid outlet defining a fluid flow path therebetween; and
a rotor disposed within the chamber.

16. The blood pump of claim 15, wherein the rotor and the curved portion of the inlet cannula defining a centrifugal force for ejecting a thrombus when disposed within the housing through the thrombus outlet.

17. The blood pump of claim 15, wherein the thrombus outlet is transverse to the cylindrical portion of the inlet cannula.

18. The blood pump of claim 15, wherein the thrombus outlet includes an extension member extending from the inlet cannula and defining a thrombus aperture.

19. The blood pump of claim 15, wherein the curved portion of the inlet cannula defines an inlet facing away from the thrombus outlet.

20. A thrombus clearing implantable blood pump comprising:
a housing including:
an inlet cannula and an outlet opposite the inlet cannula, the inlet cannula and the outlet defining a flow path therebetween, and the inlet cannula including a sidewall defining a circumference, a proximal portion, and a distal portion opposite the proximal portion, the distal portion being proximate the outlet, and the proximal portion defining a plurality of apertures extending through the sidewall and around the circumference;
an inner tube disposed within the inlet cannula, and the proximal portion of the inlet cannula extending beyond the inner tube away from the outlet; and
a rotor and a stator disposed within the housing, the stator being configured to rotate the rotor when a current is applied to the stator, the rotor and the stator defining a centrifugal force for expelling a thrombus through at least one of the plurality of apertures when the thrombus is within the blood pump.
